(19) United States
(12) Patent Application Publication
(10) Pub. No.: US 2011/0137222 A1
(43) Pub. Date: Jun. 9, 2011

(54) HEALING WOUND BANDAGING KIT AND METHOD

(76) Inventor: Michael A. Masini, Ann Arbor, MI (US)

(21) Appl. No.: 12/967,511

(22) Filed: Dec. 14, 2010

Related U.S. Application Data
(63) Continuation-in-part of application No. 29/336,018, filed on Apr. 27, 2009, now Pat. No. D629114.
Application No. 29/336,018 is a continuation of application No. 29/252,063, filed on Jan. 18, 2006, now abandoned.

(51) Int. Cl. A61F 13/00

(52) U.S. Cl. 602/43

(57) ABSTRACT

A wound healing bandaging kit includes a plurality of bandages, each with a flexible layer having a patient-contacting-surface and an outer surface. The outer surface on one of the bandages in the kit includes visual indicia portraying a neutral or negative message, and the outer surface on one of the bandages in the kit includes visual indicia portraying of a positive message. In the preferred embodiment, the visual indicia of a neutral or negative message includes the graphical image of a person's face with a neutral expression or a frown, and the visual indicia of a positive message includes the graphical image of a person's face with a smile. A method of treating a wound includes the steps of providing a wound healing bandaging kit with a plurality of bandages, one of which portrays a neutral or negative message, and another of which portrays a positive message. The bandage with the visual indicia portraying of a neutral or negative message is applied initially after a cut, bruise, surgical incision or trauma; the bandage with the visual indicia portraying a positive message is then applied after the cut, bruise, surgical incision or trauma has had an opportunity to heal.

### REFERENCE TO RELATED APPLICATIONS

This application is a continuation-in-part of U.S. Design Patent Application Ser. No. 29/336,018, filed Apr. 27, 2009, which is a continuation of U.S. Design Patent Application Ser. No. 29/252,063, filed Jan. 18, 2006, now abandoned, the entire content of each being incorporated herein by reference.

### FIELD OF THE INVENTION

This invention relates generally to wound dressings and, in particular, to a bandaging system or kit that is used while a wound heals.

### BACKGROUND OF THE INVENTION

Healing wounds after trauma and surgery can be painful for weeks in some cases. A bandaging system that makes a person feel better about the process would be welcomed by patients, especially children.

### SUMMARY OF THE INVENTION

This invention relates generally to wound dressings and more particularly resides in a bandaging system or kit that is used while a wound heals. A wound healing bandaging kit according to the invention includes a plurality of bandages, each with a flexible layer having a patient-contacting-surface and an outer surface. The outer surface on one of the bandages in the kit includes visual indicia portraying a neutral or negative message, and the outer surface on one of the bandages in the kit includes visual indicia portraying of a positive message. In the preferred embodiment, the visual indicia of a neutral or negative message includes the graphical image of a person's face with a neutral expression or a frown, and the visual indicia of a positive message includes the graphical image of a person's face with a smile.

The bandages may be generally rectangular or square, circular or oval, shaped like a U.S. Air Force shape, may have tapered ends, or may be generally hexagonal or H-shaped. The bandages may be primarily yellow in color.

A method of treating a wound according to the invention includes the steps of providing, a wound healing bandaging kit with a plurality of bandages, one of which portrays a neutral or negative message, and another of which portrays a positive message. The method continues with applying the bandage with the visual indicia portraying of a neutral or negative message initially after a cut, bruise, surgical incision or trauma, then applying the bandage with the visual indicia portraying a positive message after the cut, bruise, surgical incision or trauma has had an opportunity to heal.

### BRIEF DESCRIPTION OF THE DRAWINGS

FIG. 1 shows a bandage according to the invention including graphical indicia expressing a negative emotion, in this case a face with a frown;

FIG. 2 shows a bandage according to the invention including graphical indicia expressing a neutral emotion, in this case a face with a mouth in the form of a horizontal line;

FIG. 3 shows a bandage according to the invention including graphical indicia expressing a negative emotion, in this case a face with a smile;

FIG. 4 is a drawing the shows the backside of one of the bandages of FIGS. 1-3;

FIG. 5 is an alternative embodiment of the invention utilizing a generally square shape;

FIG. 6 is an alternative embodiment of the invention utilizing a “U.S. Air Force” shape;

FIG. 7 depicts an alternative embodiment of the invention utilizing a generally tapered shape which may form a stylized hexagon;

FIG. 8 is a drawing that shows an alternative embodiment of the invention utilizing an “H” shape; and

FIG. 9 is an illustration that shows an alternative embodiment of the invention utilizing a circular or oval shape.

### DETAILED DESCRIPTION OF THE INVENTION

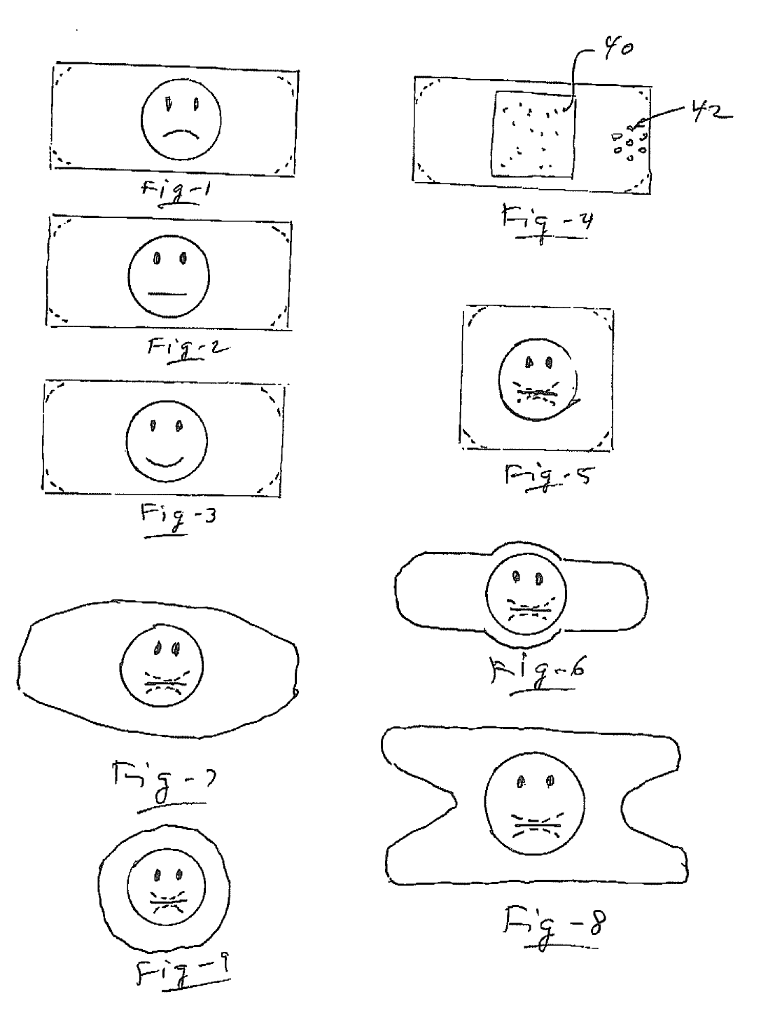

This invention resides in bandaging kits, systems and methods. FIG. 1 shows a bandage according to the invention including graphical indicia expressing a negative emotion, in this case a face with a frown. FIG. 2 shows a bandage according to the invention including graphical indicia expressing a neutral emotion, in this case a face with a mouth in the form of a horizontal line, and FIG. 3 shows a bandage according to the invention including graphical indicia expressing a negative emotion, in this case a face with a smile. The bandages of FIGS. 1-3 are generally rectangular, and may have rounded corners resulting shapes identical to those shown in U.S. Design Patent Application Ser. No. 29/336,018, incorporated herein by reference. The bandages may be any color but are preferably yellow as used with “smiley faces.”

FIG. 4 is a drawing the shows the backside of one of the bandages of FIGS. 1-3. As with all of the embodiments disclosed herein, the backside includes a fluid-absorbent portion 40 that may be entirely surrounded by a patient-contacting surface with an adhesive. The layer to which the fluid-absorbent portion 40 is attached may be fluid-impermeable, breathable, or may including perforations 42. The fluid-absorbent portion 40 may be covered with a fluid-permeable non-stick layer. All of the embodiments may have packaging (not shown) with one or more release layers which are taken of and discarded when the dressings are applied.

FIG. 5 is an alternative embodiment of the invention utilizing a generally square shape which again, may or may not have rounded corners. A neutral message is depicted, with smile and frown lines being rendered in broken-line form. FIG. 6 is an alternative embodiment of the invention utilizing a “U.S. Air Force” shape. FIG. 7 depicts an alternative embodiment of the invention utilizing a generally tapered shape which may form a stylized hexagon. FIG. 8 is a drawing that shows an alternative embodiment of the invention utilizing an “H” shape, and FIG. 9 is an illustration that shows an alternative embodiment of the invention utilizing a circular or oval shape. The invention is not limited in terms of size, and other shapes may alternatively be used.

In accordance with the invention, a plurality of bandages expressing different feelings are provided in kit form. For example, the bandages of FIGS. 1-3 may be provided in kit form, with the bandage of Figure being applied immediately after a surgical operation, trauma, or other procedure resulting in pain. The patient would wear the bandage of FIG. 1 until partialy healing has taken place, with the bandage of FIG. 2 being used to indicate that progress is being made in the healing process and at least some of the pain has subsided. When the wound is nearly healed, the bandage of FIG. 3 may be used to show that the ordeal is almost over and the pin is gone or nearly gone. Especially in the case of children, the use a progression of facial expressions may help take the child's mind off of the incision and give them something to talk about or “be proud of.”

Note that in some cases, fewer bandages may be used in accordance with the invention. For example, again using FIGS. 1-3, the caregiver may begin with a neutral expression (FIG. 2) as opposed to frowning face, then proceed right to the smiling face of FIG. 3 after sufficient healing has occurred. A kit with only two bandages may be appropriate, for example, with less invasive surgeries or smaller cuts or bruises.

I claim:

1. A wound healing bandaging kit, comprising:
a plurality bandages, each with a flexible layer having a patient-contacting-surface and an outer surface; and wherein:
the outer surface on one of the bandages in the kit includes visual indicia portraying a neutral or negative message, and
the outer surface on one of the bandages in the kit includes visual indicia portraying of a positive message.

2. The bandaging kit of claim 1, wherein:
the visual indicia of a neutral or negative message includes the graphical image of a person's face with a neutral expression or a frown; and
the visual indicia of a positive mesage includes the graphical image of a person's face with a smile.

3. The bandaging kit of claim 1, wherein the bandages are generally rectangular or square.

4. The bandaging kit of claim 1, wherein the bandages are generally circular or oval.

5. The bandaging kit of claim 1, wherein the bandages have a U.S. Air Force shape.

6. The bandaging kit of claim 1, wherein the bandages have tapered ends.

7. The bandaging kit of claim 1, wherein the bandages are generally hexagonal.

8. The bandaging kit of claim 1, wherein the bandages are generally H-shaped.

9. The bandaging kit of claim 1, wherein the outer surfaces of the bandages are primarily yellow in color.

10. A method of treating a wound, comprising the steps of:
providing the kit of claim 1;
applying the bandage with the visual indicia portraying of a neutral or negative message initially after a cut, bruise, surgical incision or trauma; and
applying the bandage with the visual indicia portraying a positive message after the cut, bruise, surgical incision or trauma has had an opportunity to heal.
